(19) United States
(12) Patent Application Publication
(10) Pub. No.: US 2009/0171415 A1
(43) Pub. Date: Jul. 2, 2009

(54) COATED MEDICAL LEADS AND METHOD FOR PREPARATION THEREOF

(76) Inventors: Kenneth Dowling, Bro (SE); Marie Herstedt, Stockholm (SE)

Correspondence Address: SCHIFF HARDIN, LLP;PATENT DEPARTMENT, 6600 SEARS TOWER, CHICAGO, IL, 60606-6473, US

(21) Appl. No.: 12/298,111

(22) PCT Filed: Apr. 27, 2006
(86) PCT No.: PCT/SE2006/000513

§ 371 Date: Oct. 22, 2008

(51) Int. Cl. A61N 1/36; B05D 3/00

(52) U.S. Cl. 607/36; 427/2.24

(57) ABSTRACT

An implantable medical device can be made more durable and long-lasting by providing a silane coating on at least a portion of a metal or metal alloy outer surface of an electrically conductive device, upon which silane coating is placed an insulating layer.

### BACKGROUND OF THE INVENTION

1. Field of the Invention

The invention relates generally to the field of coatings. More specifically, the invention relates to coatings on leads which shelter polymeric electrical insulation adjacent thereto from metal and metal ions in the lead.

2. Description of the Prior Art

Medical devices and components thereof often embody significant amounts of metal or metal alloys. While metals are typically selected based on their biocompatibility, it is often the case that structural demands on the device require that materials which are not entirely biocompatible or which are not entirely compatible with other components of the device, particularly non-metal components, are employed. One example of this is in the field of cardiac pacemakers, where electrically conductive leads extend from the pacemaker to the heart of the recipient and comprise metal lead bodies with electrical insulation along their length.

In cardiac pacemaker lead bodies, the metals used are chosen based on a number of factors. Among these is fatigue-resistance; cobalt is known to improve the fatigue-resistance to an acceptable degree. The insulating materials are also carefully chosen and flexible polymer compositions, for example, polyether-based polyurethane are commonly used. One problem which can arise from this combination, though, is that the polymer insulation rapidly degrades if it is in direct contact with metal, particularly cobalt, and/or reactive species produced in the lead body. This catalytic degradation of the polymer insulation can lead to device failure or injury to the patient, both of which should be avoided whenever possible.

The degradation phenomenon is referred to as metal ion-induced oxidation or MIO. Some solutions to the problem of improving compatibility between metal-containing devices and insulating layers, i.e., ways to prevent or reduce MIO, have been disclosed. For example, a barrier formed of a material such as polytetrafluoroethylene (PTFE, a.k.a. TEFLON) or ethylene tetrafluoroethylene (ETFE) can be placed over the device or along the inside of the insulating layer to shield the insulation from the metal or metal alloys of the device. This process does have drawbacks; the addition of a third component in the device increases its size and makes the manufacturing process more complex and costly. Furthermore, creating bonds and joints in the resultant three-layer component is more complex.

With particular regard to cardiac pacemaker leads, researchers face intense pressure to maintain or even reduce the small diameter of present electrical leads, meaning solutions that increase size are avoided whenever possible.

One way to reduce MIO without making drastic increases in final product dimensions is to provide a layer of conductive metal on the lead body, which metal is tolerated by the insulator. An example is platinum. This increases the overall dimensions by a lesser degree and typically does not present new issues during joining and bonding, however, the additional complex process steps to provide the layer and the materials themselves tend to be prohibitively expensive for many applications.

To meet the strict size demands and minimize manufacturing costs, other solutions have been proposed. For example, a voltage stabilizing additive (VSA) can be added to a polymeric insulating layer as taught in U.S. Pat. No. 6,879,861, however such polymers can be difficult and expensive to produce and the VSAs incorporated therein can leak into the patient, causing injury.

Alternately, two layers of polymers with different characteristics can be used, the layer closest the metal being selected from those which are particularly resistant to MIO, while the outer layer is chosen based on qualities such as insulating ability and promotion of sliding. U.S. Pat. No. 5,375,609, among others, provides examples of this configuration. While this might provide improved MIO-resistance in the outer insulating layer, the product cannot be optimized as regard must be given to the first layer, which may make the overall device less flexible, thicker, etc.

To help address the issues of flexibility, the inner layer of insulation can be a silicone layer, see U.S. Pat. No. 5,628,774. However, this results in a device which still has undesirably large dimensions.

Thus, despite the advances described herein, there remains a need in the art to improve upon known techniques and optimally provide improved devices which reduce the stress on insulators without compromising device dimensions or other beneficial properties such as flexibility.

### SUMMARY OF THE INVENTION

It is an object of the present invention to provide improved implantable devices which comprise an electrically conductive metal or metal alloy having an outer surface, a silane coating disposed on at least a portion of the metal or metal alloy outer surface, and an insulating layer having an inner surface configured to fit over at least part of the silane coating. The electrically conductive metal or metal alloy can be an elongated lead, such as a cardiac pacemaker lead.

The electrically conductive metal or metal alloy can comprise cobalt, and/or the insulating layer can be polymer-based, such as polyurethane. The silane coating can be sputter coated. The silane coating can further be oxidized, cross-linked, or reduced.

According to a further embodiment of the invention, a method of preparing an insulated conductor is provided which comprises providing an elongated metal or metal alloy conductor having an outer surface, coating at least a portion of the conductor with a silane coating, and providing an insulation layer around at least a portion of the silane coating.

The coating can be effected through chemical vapour deposition or plasma deposition of a vapour phase silane, alternatively, it can comprise dipping in a liquid phase silane. The silane coating can be oxidised, cross-linked, or reduced prior to the providing an insulation layer step. The insulation layer can be provided around the entire silane coating.

According to a further embodiment of the invention, a method of protecting polymeric electrical insulation from metal ion-induced oxidation is provided, which comprises providing an electrically conductive metal or metal alloy having an outer surface, coating at least a portion of the conductive metal or metal alloy outer surface, and positioning the polymeric electrical insulation on the silane coating.

According to a further embodiment of the invention, a kit for preparing an implantable medical device is provided, which comprises an electrically conductive metal or metal alloy, a means to place a silane coating on the metal or metal alloy, and an insulating layer configured to fit on the electrically conductive metal or metal alloy.

As used herein, “silane” refers to any molecule having the molecular formula RSiX₍₄₋ₙ₎. Cyclosilanes as well as branched silanes are included in the definition. Silanes comprise silicone, an organic functional group (“R”) such as a vinyl, amino, chloro, epoxy or mercapto group, and a second functional group (“X”) such as a methoxy or ethoxy group. The R group attaches to an organic resin while the X group attaches to inorganic material or substrates, having a coupling effect. The X group can hydrolyze to produce silanol, which forms a metal hydroxide or siloxane bond with inorganic material, while the R group of the silane molecule reacts with organic material to produce a covalent bond.

Examples of silanes which can be used in conjunction with the present invention include, but are not limited to: Tetraethoxysilane (TEOS), Tetramethoxysilane (TMOS), Vinyltriethoxysilane, Vinyltrimethoxysilane, Trimethylchlorosilane, Dimethylvinylchlorosilane, n-Octyltrichlorosilane, Trimethylmethoxysilane, Methyltrimethoxysilane, Ethyltrimethoxysilane, Propyltrimethoxysilane, i-Butyltrimethoxysilane, Methacryloxypropyltrimethoxysilane, and hydrogen-terminated Dimethyl siloxane.

### DESCRIPTION OF THE PREFERRED EMBODIMENTS

As noted above, the present invention provides a new configuration for implantable medical devices which allows for a slim profile while protecting insulating layers from MIO. This is achieved by providing a silane coating on at least part of the metal or metal alloy containing device. This silane coating shields the overlying insulating layer from MIO while having a negligible effect on the dimensions and mechanical properties of the device.

Silane coatings and methods of applying the same are known in the art. For example, sputter coating or sputter deposition involves placing a substrate, such as a medical device, in a processing chamber adjacent to a sputtering cathode target, which serves as a source of coating material such as silane. The pressure in the processing chamber, which is usually filled with an inert gas such as argon, is then reduced to a near vacuum, and a negative voltage is applied to the target. During this process atoms or small particles of the target material are discharged and move across the chamber until they strike the surface of the substrate, where they adhere to the surface and form a thin film or coating layer thereon. To coat only a portion of a device it can be partially masked in the chamber or only the portion to be coated can be present in the chamber and later connected to a non-coated portion.

An alternative to sputter coating is vapor deposition, where a reactant vapor or vapor mixture is brought into contact with a surface on which a thin film is deposited. For reasonable reaction rates the substrate is preferably heated to relatively high temperatures. If the medical device to be coated according to the present invention is sensitive to elevated temperatures, it is also possible to provide a silane coating through plasma deposition. Plasma deposition involves supplying energy to the reactant, such as silane, by an electrical discharge in a gas which forms plasma in the deposition chamber. The substrate can then be immersed in the plasma. Relatively low temperatures can be employed and result in a thin film on the substrate. Yet another option is to dip the device in liquid phase silane.

The resultant coated device has a layer of silane that is extremely thin, in the area of 10 nm to 10 μm. This allows the present invention to provide a device which does not measurably exceed current product dimensions. Furthermore, the coated device maintains the advantageous properties of the underlying material, whether those are strength, flexibility, or other.

By treating the silane layer after deposition, improved properties such as a more strongly-adhered coating, altered mechanical strength or wear resistance, or a change in ion barrier characteristics can be achieved. For example, the coating can be oxidized through, for example, injecting a pulse of oxygen gas after deposition of the silane layer in order to form a fully oxidized film. Another treatment is to cross-link the silane layer, which can be accomplished through the use of any of the many known cross-linking agents such as high energy irradiation or peroxide treatment. The silane coating may also be reduced to improve the properties of the silane layer.

The devices and methods of the present invention are particularly effective at shielding polyether-based polyurethane insulations from cobalt present in cardiac pacemaker leads. This is both because the silane coating effectively traps the metal ions in the lead body, thus avoiding the problem of cobalt-induced degradation of the polyurethane, but also because the silane coating does not have a significant effect on product dimensions and mechanical properties, two factors that are exceedingly important with cardiac pacemaker leads.

### Example 1

### Cardiac Pacemaker Lead

A cardiac pacemaker lead body is formed according to known methods from the fatigue-resistant electrical conducting material MP35N (a nonmagnetic, nickel-cobalt-chromium-molybdenum alloy available from Carpenter Technologies, Reading, Pa.). TEOS is coated onto the complete outer surface of the lead body using sputter coating methods known in the art. The resultant coated lead body is oxidized using pure oxygen gas and inserted into a commercially available polyethylene tubular insulation having the appropriate diameter. The resultant coated, insulated lead can be connected to a cardiac pacemaker at a proximal end, inserted into a patient and connected to the patient's heart at a distal end.

The lead discussed herein offers improved resistance to degradation of the polymer insulation without possessing any statistically significant increase in product dimension. Furthermore, the flexibility, fatigue-resistance, glidability and other beneficial properties of the insulated lead are maintained. The silane coating on the lead thus provides the additional benefit of extending potential product life. Extending product life in a product such as a pacemaker lead reduces the risk of complications or injury to the patient while also reducing the chance that an additional procedure is required to remove and replace a lead, which also reduces the risk of adverse outcome for the patient while minimizing medical treatment costs.

Although modifications and changes may be suggested by those skilled in the art, it is the intention of the inventors to embody within the patent warranted heron all changes and modifications as reasonably and properly come within the scope of their contribution to the art.

We claim as our invention:

1. An implantable medical device, comprising:
an electrically conductive metal or metal alloy having an outer surface;
a silane coating disposed on at least a portion of the metal or metal alloy outer surface; and
an insulating layer having an inner surface configured to fit over at least part of said silane coating.

2. A device according to claim 1, wherein the electrically conductive metal or metal alloy is an elongated lead

3. A device according to claim 2, wherein the elongated lead is a cardiac pacemaker lead.

4. A device according to claim 1, wherein the electrically conductive metal or metal alloy comprises cobalt.

5. A device according to claim 1, wherein the silane coating is a sputter coated silane coating.

6. A device according to claim 1, wherein the silane coating is oxidized, cross-linked, or reduced.

7. A device according to claim 1, wherein the insulating layer is polymer-based.

8. A device according to claim 7, wherein the polymer is polyurethane.

9. A method of preparing an insulated conductor, comprising:
providing an elongated metal or metal alloy conductor having an outer surface;
coating at least a portion of said conductor with a silane coating; and
providing an insulation layer around at least a portion of said silane coating.

10. A method according to claim 9, wherein said coating step comprises chemical vapour deposition or plasma deposition of a vapour phase silane.

11. A method according to claim 9, wherein said coating step comprises dipping in a liquid phase silane.

12. A method according to claim 9, further comprising oxidising, cross-linking, or reducing the silane coating prior to the providing an insulation layer step.

13. A method according to claim 9, wherein the insulation layer is provided around the entire silane coating.

14. A method of protecting polymeric electrical insulation from metal ion-induced oxidation, comprising:
providing an electrically conductive metal or metal alloy having an outer surface;
coating at least a portion of said conductive metal or metal alloy outer surface; and
positioning the polymeric electrical insulation on the silane coating.

15. A kit for preparing an implantable medical device, comprising:
an electrically conductive metal or metal alloy;
a means to place a silane coating thereon; and
an insulating layer configured to fit on the electrically conductive metal or metal alloy.
